SEQUENCE LISTING (8 sequences)

SEQ ID NO: 1 - skipped

SEQ ID NO: 2 - DNA, 70 bp, synthetic construct
  source 1..70: mol_type = other DNA
aatgatacggcgaccaccgagatctacacctagcgctacactctttccctacacgacgctcttccgatct

SEQ ID NO: 3 - DNA, 66 bp, synthetic construct
  source 1..66: mol_type = other DNA
caagcagaagacggcatacgagataaccgcgggtgactggagttcagacgtgtgctcttccgatct

SEQ ID NO: 4 - DNA, 53 bp, synthetic construct
  source 1..53: mol_type = other DNA
acacgacgctcttccgatctnnnnnnnnnntgccagttaacgtcttccttctc

SEQ ID NO: 5 - DNA, 52 bp, synthetic construct
  source 1..52: mol_type = other DNA
gacgtgtgctcttccgatctnnnnnnnnnnccacacagcaaagcagaaactc

SEQ ID NO: 6 - DNA, 158 bp, synthetic construct
  source 1..158: mol_type = other DNA
tgccagttaacgtcttccttctctctctgtcatagggactctggatcccagaaggtgagaaagttaaaattcccgtcgctatcaaggaattaagagaagcaacatctccgaaagccaacaaggaaatcctcgatgtgagtttctgctttgctgtgtgg

SEQ ID NO: 7 - DNA, 218 bp, synthetic construct
  source 1..218: mol_type = other DNA
acacgacgctcttccgatctnnnnnnnnnntgccagttaacgtcttccttctctctctgtcatagggactctggatcccagaaggtgagaaagttaaaattcccgtcgctatcaaggaattaagagaagcaacatctccgaaagccaacaaggaaatcctcgatgtgagtttctgctttgctgtgtggnnnnnnnnnnagatcggaagagcacacgtc

SEQ ID NO: 8 - DNA, 314 bp, synthetic construct
  source 1..314: mol_type = other DNA
aatgatacggcgaccaccgagatctacacctagcgctacactctttccctacacgacgctcttccgatctnnnnnnnnnntgccagttaacgtcttccttctctctctgtcatagggactctggatcccagaaggtgagaaagttaaaattcccgtcgctatcaaggaattaagagaagcaacatctccgaaagccaacaaggaaatcctcgatgtgagtttctgctttgctgtgtggnnnnnnnnnnagatcggaagagcacacgtctgaactccagtcacccgcggttatctcgtatgccgtcttctgcttg

(19) United States
(12) Patent Application Publication
(10) Pub. No.: US 2026/0218287 A1
(43) Pub. Date: Jul. 30, 2026

(54) METHOD FOR SIMULTANEOUS MULTIPLEX DETECTION OF MULTIPLE CANCER-ASSOCIATED ALTERATION AND DETERMINATION OF TISSUE-OF-ORIGIN

(71) Applicant: Lucence Life Sciences PTE. LTD., Singapore (SG)

(72) Inventors: Michelle PEK, Singapore (SG); Hao CHEN, Singapore (SG); Zi Yi WAN, Singapore (SG); Kao Chin NGEOW, Singapore (SG); Min-Han TAN, Singapore (SG); Jonathan POH, Singapore (SG)

(21) Appl. No.: 19/159,094

(22) PCT Filed: Dec. 29, 2023
(86) PCT No.: PCT/SG2023/050865

§ 371 Date: Aug. 22, 2025

(30) Foreign Application Priority Data
Feb. 24, 2023 (SG) 10202300487P

(51) Int. Cl. C12Q 1/686; C12Q 1/48; C12Q 1/6869; C12Q 1/6886; C40B 40/06; C40B 50/06; C40B 70/00; G16B 30/10; G16H 50/70

(52) U.S. Cl. CPC C12Q 1/686; C12Q 1/48; C12Q 1/6869; C12Q 1/6886; C12Y 207/07; C40B 40/06; C40B 50/06; C40B 70/00; G16B 30/10; G16H 50/70; C12Q 2600/112; C12Q 2600/118; C12Q 2600/156; C12Q 2600/16

(57) ABSTRACT

Disclosed is a method of detecting one or more cancer-associated alterations and/or determining site of origin of a cancer-associated alteration using nucleic acid from a biological sample. The method comprises (a) extracting nucleic acid from the biological sample; (b) performing multiplexed PCR reactions on the nucleic acid using: a plurality of target capture primer pairs specific to a plurality of target genes comprising one or more cancer-associated alteration, wherein each target capture primer pair comprises a forward primer and a reverse primer, each comprising a target-specific sequence at its 3′ end, a barcode sequence linker, and an adapter-specific sequence at its 5′ end; (c) sequencing the plurality of amplicons from (b) to obtain a plurality of sequencing reads; and (d) comparing the plurality of sequencing reads with one or more pre-determined reference profile to compute one or more likelihood score, which indicates the probability of a cancer-associated alteration originating from a specific site of origin. Also disclosed is a kit for said method.

### FIELD OF INVENTION

The present disclosure relates to the use of highly multiplexed amplicon-based target sequencing to simultaneously detect multiple cancer-associated alterations and tissue-of-origin (TOO) in nucleic acids isolated from biological samples. In particular, the present invention relates to the simultaneous detection of multiple cancer-associated alterations and determination site of origin of a cancer-associated alteration in cell-free DNA (cfDNA) and genomic DNA (gDNA) present in blood samples.

### BACKGROUND

The majority (78%) of cancer deaths in Asia arise from cancers with no existing screening recommendations. Earlier cancer detection is associated with improved outcomes. While conventional single-cancer screening by liquid biopsy is recognized in Asia, there is currently no recognized screening assay for multi-cancer screening (MCS) by liquid biopsy. Blood-based multi-cancer early detection (MCED) testing has emerged as a promising paradigm for earlier detection of cancer, which is associated with better outcomes. However, little is known about the real-world performance of such assays in Asia.

In addition, TOO prediction has proven to be valuable in aiding identification of the exact tumor site for confirmatory diagnosis, which may lead to better prognosis.

As such, there is a need to provide a method for sensitive and accurate detection of multiple types of cancer at an early stage of cancer progression to improve cancer diagnosis and prognosis. There is a need to provide a method to detect multiple cancer-associated alterations simultaneously and determine the site of origin of a cancer-associated alteration using liquid biopsy as starting material.

### SUMMARY

In a first aspect, the present disclosure refers to a method of detecting one or more cancer-associated alterations and/or determining site of origin of a cancer-associated alteration within a biological sample comprising nucleic acids, comprising the steps of:
(a) extracting nucleic acid from the biological sample obtained from a subject;
(b) performing a plurality of multiplexed PCR reactions on the nucleic acid using:
   a plurality of target capture primer pairs specific to a plurality of target genes comprising one or more cancer-associated alteration,
   wherein each target capture primer pair comprises a forward target capture primer and a reverse target capture primer specific to a target gene comprising one or more cancer-associated alteration,
   wherein each forward target capture primer and each reverse target capture primer comprises a target-specific sequence at its 3′ end that is specific to the target gene, a barcode sequence linker, and an adapter-specific sequence at its 5′ end,
   thereby generating a plurality of amplicons comprising a plurality of target gene sequences corresponding to the plurality of target genes comprising one or more cancer-associated alteration;
(c) sequencing the plurality of amplicons from step (b) to obtain a plurality of sequencing reads, wherein optionally, the sequencing is multiplex sequencing on a next-generation sequencing platform; and
(d) comparing the plurality of sequencing reads comprising one or more cancer-associated alteration with one or more pre-determined reference cancer-associated alteration profile to compute one or more likelihood score, wherein the one or more likelihood score indicates the probability of a cancer-associated alteration originating from a specific site of origin,
to thereby detect the presence of one or more cancer-associated alterations and/or determine the site of origin of a cancer-associated alteration.

In a second aspect, the present disclosure refers to a kit for detecting one or more cancer-associated alterations and/or determining site of origin of a cancer-associated alteration within a biological sample according to the method of the first aspect, comprising a plurality of target capture primer pairs specific to a plurality of target genes comprising one or more cancer-associated alteration as defined in step (b) of the first aspect, and instructions for use in the method of the first aspect.

### BRIEF DESCRIPTION OF DRAWINGS

The invention will be better understood with reference to the detailed description when considered in conjunction with the non-limiting examples and the accompanying drawings, in which:

FIG. 1 is an overview of the experimental workflow of the MCS testing method described herein.

FIG. 2 is an overview of the TOO prediction model of the MCS testing method described herein. The inhouse developed weighted sum model was used to predict the primary site of a tumor. The model was trained using public databases and expert curation data. The weights assigned to each alteration and its co-occurring combinations represented the importance or contribution of each alteration to each potential TOO.

FIG. 3 shows an overview of the MCS assay performance for detection of cancer-associated mutations, as well as TOO prediction accuracy. The sensitivity of detection of cancer associated alterations was evaluated in a cohort of 476 untreated cancer patients and the specificity of assay was evaluated in a cohort of 137 controls. A weighted sum model was used for single site TOO prediction in an expanded cohort of 1478 samples with a clear single cancer diagnosis in 10 cancer types.

FIG. 4 (comprised of FIGS. 4A and 4B) shows the sensitivity and specificity of cancer-associated alterations detection in MCS assay. FIG. 4A shows the distribution of cancer samples by cancer stage (localized, metastatic, unknown) detected in the testing cohort. FIG. 4B shows the distribution of cancer type detected in the testing cohort.

FIG. 5 shows that variant allele frequency (VAF) of cancer-associated alterations was higher in metastatic cancer cases detected by the MCS assay.

FIG. 6 (comprised of FIGS. 6A and 6B) shows the prediction results of the TOO prediction method described herein. FIG. 6A is a pie chart showing the distribution of cancer types in an expanded 1478-sample cohort obtained from the TOO prediction model described herein. FIG. 6B is a dot plot showing the number of cancer-associated alterations per subject sample used for TOO prediction. The median number of cancer-associated alterations used for TOO predictions ranged from one to three alterations per cancer type cohort.

FIG. 7 is a line graph demonstrating the improvement of overall TOO prediction accuracy with inclusion of clinico-demographic data.

FIG. 8 are line graphs illustrating TOO prediction accuracy and reporting rate for specific cancer types. Improved TOO prediction was observed in seven out of 10 cancer types (improvement of 2.18-50.0% attributable to the use of co-occurrence and clinic-demographic data). TOO reporting rate remained largely consistent in most cancer types.

FIG. 9 is an overview of the MCED assay workflow described herein. Targeted ultrasensitive amplicon-based next-generation sequencing (NGS) was used to detect cancer signal (CS) status via the presence of cancer-associated mutations and viral DNA in 84 genes in plasma cell-free DNA (cfDNA). Matched white blood cell DNA was analyzed to exclude clonal hematopoiesis as a contributor.

FIG. 10 is a flowchart showing the consecutive results from 264 subjects (1/2023-6/2023) from Singapore, Hong Kong and Malaysia that underwent testing at a CAP-accredited, CLIA-certified laboratory.

FIG. 11 is a bar graph showing the baseline population characteristics of the 264 subjects that underwent testing. The mean age was 55.1 years with 61.4% being males.

FIG. 12 is a dot plot illustrating the maximum plasma VAF per sample tested. 37.5% of the subjects (99/264) had variants from clonal hematopoiesis (CH)/CHIP detected in buffy coat gDNA only (n=61) or both buffy coat gDNA and plasma cfDNA (n=38). In plasma cfDNA, VAF of cancer-associated alterations ranged from 0.42%-4.66% and CH/CHIP ranged from 0.02%-6.65%.

### DETAILED DESCRIPTION

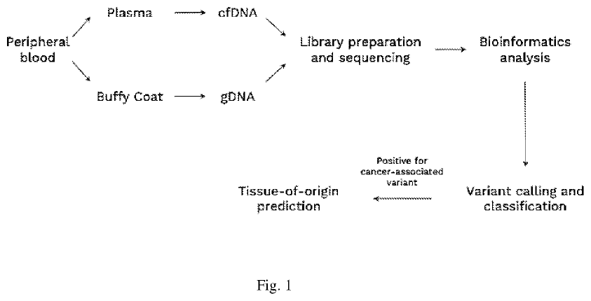

The present disclosure describes a circulating tumor DNA (ctDNA) mutation-based testing workflow based on targeted amplicon-based NGS testing of plasma and matched buffy coat with a custom pipeline incorporating clinico-demographic factors for TOO calling (FIG. 1).

In a first aspect, the present disclosure refers to a method of detecting one or more cancer-associated alterations and/or determining site of origin of a cancer-associated alteration within a biological sample comprising nucleic acids, comprising the steps of:
(a) extracting nucleic acid from the biological sample obtained from a subject;
(b) performing a plurality of multiplexed PCR reactions on the nucleic acid using:
   a plurality of target capture primer pairs specific to a plurality of target genes comprising one or more cancer-associated alteration,
   wherein each target capture primer pair comprises a forward target capture primer and a reverse target capture primer specific to a target gene comprising one or more cancer-associated alteration,
   wherein each forward target capture primer and each reverse target capture primer comprises a target-specific sequence at its 3′ end that is specific to the target gene, a barcode sequence linker, and an adapter-specific sequence at its 5′ end,
   thereby generating a plurality of amplicons comprising a plurality of target gene sequences corresponding to the plurality of target genes comprising one or more cancer-associated alteration;
(c) sequencing the plurality of amplicons from step (b) to obtain a plurality of sequencing reads, wherein optionally, the sequencing is multiplex sequencing on a next-generation sequencing platform;
(d) comparing the plurality of sequencing reads comprising one or more cancer-associated alteration with one or more pre-determined reference cancer-associated alteration profile to compute one or more likelihood score, wherein the one or more likelihood score indicates the probability of a cancer-associated alteration originating from a specific site of origin,
to thereby determine the presence of one or more cancer-associated alterations and/or the site of origin of a cancer-associated alteration.

In one example, prior to performing step (c) of the method of the first aspect, the method disclosed herein comprises further performing a step of removing target capture primers that have not been incorporated into the plurality of amplicons generated in step (b) of the first aspect using at least one nuclease, thereby generating a purified mixture of the plurality of amplicons comprising the plurality of target gene sequences. In one example, the at least one nuclease used to remove the target capture primers that have not been incorporated into amplicons is an endonuclease and/or an exonuclease.

In one example, the method of the first aspect further comprises the steps of:
(e) amplifying the plurality of amplicons in the purified mixture as defined herein by using universal indexed adapter primers to generate a sequencing library, wherein each amplicon of the sequencing library comprises two barcode sequences;
(f) purifying the sequencing library obtained from step (c);
(g) mapping the plurality of sequencing reads obtained from step (c) of step 1 of the method of the first aspect to a first reference genome;
(h) grouping the sequencing reads where the barcode sequences of the sequencing reads are identical into a consensus cluster;
(i) performing a sequence alignment of each consensus cluster obtained from step (h) with all consensus clusters having at least one overlapping barcode sequence, then determining the presence of a consensus base in each sequence alignment result to generate a consensus sequencing read;
(j) mapping each of the consensus sequencing read from step (i) with a second reference genome; and
(k) identifying the differences between the consensus sequencing read and the reference genome from step (j) to identify consensus sequencing reads comprising one or more cancer-associated alteration.

In one example, step (d) of the method of the first aspect further comprises optimizing the one or more likelihood score based on a clinico-demographic profile of the subject. In one example, the clinico-demographic profile comprises nationality, gender, age, or a combination thereof. One example of an optimization and/or adjustment of the one or more likelihood score is-if the subject is female, cancers which are only found in males are excluded from the TOO prediction model (e.g., prostate, testis, penile cancers) and vice versa (ovarian, cervical, vulvar cancers are excluded for male subjects). Another example of an optimization and/or adjustment of the one or more likelihood score is based on country of occurrence of the cancers, specifically, the difference in the incidence of specific cancers in different regions. For instance, Epstein-Barr virus is known to be associated with 85% of nasopharyngeal cancers (NPC) and only 9% of stomach cancers. However, stomach cancers are much more likely to occur in countries such as Japan or South Korea (close to 20 times more likelihood of occurrence). One the other hand, NPC is endemic to countries like Singapore. Hence, the optimization and/or adjustment in the TOO prediction model was designed to take these clinico-demographic profile into account to give the final likelihood score.

In one example, the nucleic acid is selected from the group consisting of cell-free nucleic acid and genomic nucleic acid. In one example, the cell-free nucleic acid is cell-free DNA (cfDNA) or cell-free RNA (cfRNA). In one example, the genomic nucleic acid is genomic DNA (gDNA) or genomic RNA (gRNA). In one example, the cell-free nucleic acid is cfDNA and the genomic nucleic acid is gDNA. In one example, the nucleic acid comprises a combination of cfDNA and gDNA. In one example, the nucleic acid comprises cfDNA, cfRNA, gDNA, gRNA, or a combination thereof. In one example, the nucleic acid comprises cfDNA and gDNA. In one example, the nucleic acid comprises a combination of cfDNA, gDNA, and cfRNA. In one example, the nucleic acid comprises a combination of cfDNA, gDNA, cfRNA, and gRNA. In one example, the cfDNA is circulating tumor DNA (ctDNA). In one example, the gRNA is RNA from whole blood.

In one example, the one or more cancer-associated alteration is selected from a group comprising of single-nucleotide variations, insertions, deletions, genomic copy number alterations, deletions of homopolymeric regions, total mutation (or variant) load, detection of microbial DNA sequences, detection of polymorphisms or single-nucleotide variations in nucleic acid sequences, and incorporation of viral DNA. In one example, “single nucleotide variations” refer to variation in a single nucleotide that occurs at a specific position in the genome, differing from the nucleotide defining the position in the reference genome. In one example, the “insertion” is a sequence change where at least one nucleotide is inserted between two nucleotides. In one example, the “insertion” is a sequence change where more than 10 nucleotides are inserted between two nucleotides. In one example, the “insertion” is a sequence change where more than 20 nucleotides are inserted between two nucleotides. In one example, the “insertion” is a sequence change where more than 30 nucleotides are inserted between two nucleotides. In one example, the “insertion” is a sequence change where more than 40 nucleotides are inserted between two nucleotides. In one example, the “insertion” is a sequence change where more than 50 nucleotides are inserted between two nucleotides. In one example, the “insertion” may be a “small insertion” where less than 50 nucleotides are inserted between two nucleotides. In one example, the “insertion” is a “duplication”. In one example, the “duplication” is a sequence change where a copy of one or more nucleotides are inserted directly 3′-flanking of the original copy. In one example, the “deletion” is a sequence change where at least one nucleotide is removed. In one example, the “deletion” is a sequence change where more than 10 nucleotides are removed. In one example, the “deletion” is a sequence change where more than 20 nucleotides are removed. In one example, the “deletion” is a sequence change where more than 30 nucleotides are removed. In one example, the “deletion” is a sequence change where more than 40 nucleotides are removed. In one example, the “deletion” is a sequence change where more than 50 nucleotides are removed. In one example, the “deletion” may be a “small deletion” where less than 50 nucleotides are removed. In one example, the term “copy number alteration” refers to the repetition of sections of the genome (duplication) or loss of sections of the genome (deletion). In one example, the term “deletions of homopolymeric regions” refers to the shortening of a homopolymeric tracts in the genome. An example of “deletions of homopolymeric region” is GCGAAAAAAAAAAAAAAATA becomes GCGAAATA, this a deletion of 12 A's from the homopolymeric tract of 15 A's. In one example, the term “polymorphism” refers to a variation in a single nucleotide that occurs at a specific position in the genome, and is a variation in all copies of the organism's genome, differing from nucleotide defining the position in the organism's population (reference). A person skilled in the art is aware that the sum of all of the variants within the nucleic acid sequence is known as total mutation (or variant) load or tumour mutational burden (TMB). A person skilled in the art is also aware that determining the total mutation (or variant) load or tumour mutational burden (TMB) is useful in determining the therapeutic target of certain diseases (such as cancer). Microbial nucleic acid sequences are considered non-human genomic sequences and/or foreign DNA sequences. In one example, the microbial nucleic acid sequence is microbial DNA sequence. In one example, the microbial nucleic acid sequence is microbial RNA sequence. In one example, the microbial RNA sequence is SARS-COV-2 RNA sequence. In one example, the structural rearrangement may be structural rearrangement(s) in the DNA giving rise to variant RNA molecules. In one example, the structural rearrangement may be structural variants of RNA molecules. In one example, the structural rearrangement may be copy number alterations in the DNA, giving rise to variable amounts of RNA molecules. In one example, the structural rearrangements may be structural variants of RNA molecules and copy number alterations of RNA molecules. In one example, the structural arrangement may be structural rearrangement(s) in DNA molecules. In one example, the term “rearrangement” refers to rearrangement in the order of sections of the DNA, giving rise to a variant transcript of an RNA molecule. In one example, the structural rearrangement is a fusion, such as a gene fusion. In one example, the term “fusion” refers to structural variations produced through structural rearrangements, such as interchromosomal or intrachromosomal rearrangements. In one example, the structural rearrangement may include, but are not limited to, deletion, insertion (such as duplication), inversion, transversion, translocation, alternative splicing, and the like. In one example, the term “translocation” refers to rearrangement of parts between non-homologous chromosomes, which can result in “fusion”. In one example, “altered splicing” refers to aberrant splicing of a single gene transcript that may cause one or more exons in sequence to be spliced out of the RNA, bringing usually more distant exons of the same gene in juxtaposition. Altered splicing involves the same gene, compared to fusion which is a definition reserved for two genes. One example of altered splicing includes MET exon 14 skipping where exon 14 of MET gene is spliced out bringing exon 13 and exon 15 in proximity. In one example, the genomic alteration may be RNA structural variants. In another example, the genomic alteration may be copy number alterations of RNA molecules. In one example, the cancer-associated alterations include the incorporation of viral DNA into a host cell genome. In one example, the viral DNA is Epstein-Barr virus (EBV), hepatitis B virus (HBV), and/or Human papillomavirus (HPV). It is known in the art that HPV is a group of more than 200 related viruses, some of which are spread through vaginal, anal, or oral sex. Sexually transmitted HPV types fall into two groups, low risk and high risk. High-risk HPVs can cause several types of cancer, including cervical cancer, oropharyngeal cancer, anal cancer, penile cancer, vaginal cancer, and vulvar cancer. Therefore, the inclusion of primers that detect HPV genotypes with high carcinogenicity enhances cancer detection. High-risk HPV genotypes include those in Group 1 and 2A as further defined below. In one example, the HPV genotypes detectable by the method of the present disclosure comprises HPV genotypes which arc carcinogenic to humans (Group 1), HPV genotypes which are probably carcinogenic to humans (Group 2A), HPV genotypes which are possibly carcinogenic to humans (Group 2B), and HPV genotypes which are not classifiable as to its carcinogenicity to humans (Group 3). In one example, HPV genotypes which are carcinogenic to humans (Group 1) comprises HPV genotypes such as HPV16, HPV18, HPV31, HPV33, HPV35, HPV39, HPV45, HPV51, HPV52, HPV56, HPV58, and HPV59. In one example, HPV genotypes which are probably carcinogenic to humans (Group 2A) comprises HPV genotype such as HPV 68. In one example, HPV genotypes which are possibly carcinogenic to humans (Group 2B) comprises HPV genotypes such as HPV5, HPV8, HPV26, HPV30, HPV34, HPV53, HPV66, HPV67, HPV69, HPV70, HPC73, HPV82, HPV85, and HPV97. In one example, HPV genotypes which are not classifiable as to its carcinogenicity to humans (Group 3) comprises HPV genotypes such as HPV genus beta (except types 5 and 8), HPV6, and HPV11. In one example, the HPV genotypes detectable by the method of the present disclosure comprises HPV genotypes such as (but not limited to) HPV16, HPV18, HPV31, HPV33, HPV35, HPV39, HPV45, HPV51, HPV52, HPV56, HPV58, HPV59 (From Group 1 classification); HPV 68 (From Group 2A classification); and HPV26, HPV53, HPV66, HPV67, HPV70, HPC73, and HPV82 (From Group 2B classification).

In one example, the biological sample comprising nucleic acids is a liquid sample. In one example, the liquid sample is a bodily fluid. In one example, the cell-free nucleic acid (such as cfDNA) used for the present invention may be derived from bodily fluid. In one example, the bodily fluid is selected from the group consisting of blood, bone marrow, cerebral spinal fluid, peritoneal fluid, pleural fluid, lymph fluid, ascites, serous fluid, sputum, lacrimal fluid, stool, urine, saliva, ductal fluid from breast, gastric juice and pancreatic juice. In one example, the bodily fluid is blood. In one example, the blood is plasma. In another example, the blood is buffy coat.

In one example, step (a) of the method of the first aspect further comprises separating the biological sample into a plasma component and a buffy coat component, prior to extracting the cfDNA or cfRNA from the plasma component and gDNA or gRNA from buffy coat component, if the biological sample is blood. In one example, the separation is by mechanical separation. In one example, the separation is by centrifugation. In one specific example, the centrifugation is a 2-step centrifugation process: first centrifugation was done at 1600×g for 10 min at 4° C. to separate plasma. The plasma layer was transferred to a separate tube and centrifuged at 16,000×g for 10 min at 4° C. to further remove cellular contaminants. Various methods for separating the biological sample into a plasma component and a buffy coat component are known in the art and may be used for the purpose of the disclosed method.

In one example, the plurality of multiplexed PCR reactions as defined in step (b) of the method of the first aspect performed on the plasma component and the buffy coat component are independent of each other.

In one example, when the biological sample is blood, the nucleic acid to be used for step (b) of the method of the first aspect harbor genomic and/or cancer-associated alterations that arise independently from clonal hematopoiesis.

In one example, the amount of nucleic acid within the biological sample is from 1 ng to 1,200,000 ng, or from 10 ng to 1,100,000 ng, or from 100 ng to 1,000,000 ng, or from 1,000 ng to 900,000 ng, or from 10,000 ng to 800,000 ng, or from 100,000 ng to 700,000 ng, or from 200,000 ng to 600,000 ng, or from 300,000 ng to 500,000 ng, or about 1 ng, or about 10 ng, or about 20 ng, or about 30 ng, or about 40 ng, or about 50 ng, or about 60 ng, or about 70 ng, or about 80 ng, or about 90 ng, or about 100 ng, or about 110 ng, or about 120 ng, or about 130 ng, or about 140 ng, or about 150 ng, or about 160 ng, or about 170 ng, or about 180 ng, or about 190 ng, or about 200 ng, or about 220 ng, or about 230 ng, or about 240 ng, or about 250 ng, or about 260 ng, or about 270 ng, or about 280 ng, or about 290 ng, or about 300 ng, or about 400 ng, or about 500 ng, or about 600 ng, or about 700 ng, or about 800 ng, or about 900 ng, or about 1,000 ng, or about 10,000 ng, or about 100,000 ng, or about 200,000 ng, or about 300,000 ng, or about 400,000 ng, or about 500,000 ng, or about 600,000 ng, or about 700,000 ng, or about 800,000 ng, or about 900,000 ng, or about 1,000,000 ng, or about 1,100,000 ng, or about 1,200,000 ng. In one example, the amount of nucleic acid within the biological sample which can be used as starting material for the method disclosed herein is from 5 ng to 100 ng, or from 10 ng to 90 ng, or from 20 ng to 80 ng, or from 30 ng to 70 ng, or from 40 ng to 60 ng, or about 5 ng, or about 10 ng, or about 20 ng, or about 30 ng, or about 40 ng, or about 50 ng, or about 60 ng, or about 70 ng, or about 80 ng, or about 90 ng, or about 100 ng. In one example, the cell-free nucleic acid extracted from the biological sample is from Ing to 900,000 ng. In one example, the genomic nucleic acid extracted from the biological sample is from 10 ng to 300,000 ng. In one example, the amount of cfDNA extracted from plasma is Ing to 900,000 ng, or from 10 ng to 890,000 ng, or from 100 ng to 800,000 ng, or from 1,000 ng to 700,000 ng, or from 10,000 ng to 600,000 ng, or from 100,000 ng to 500,000 ng, or from 200,000 ng to 400,000 ng, or about 1 ng, or about 10 ng, or about 20 ng, or about 30 ng, or about 40 ng, or about 50 ng, or about 60 ng, or about 70 ng, or about 80 ng, or about 90 ng, or about 100 ng, or about 110 ng, or about 120 ng, or about 130 ng, or about 140 ng, or about 150 ng, or about 160 ng, or about 170 ng, or about 180 ng, or about 190 ng, or about 200 ng, or about 220 ng, or about 230 ng, or about 240 ng, or about 250 ng, or about 260 ng, or about 270 ng, or about 280 ng, or about 290 ng, or about 300 ng, or about 400 ng, or about 500 ng, or about 600 ng, or about 700 ng, or about 800 ng, or about 900 ng, or about 1,000 ng, or about 10,000 ng, or about 100,000 ng, or about 200,000 ng, or about 300,000 ng, or about 400,000 ng, or about 500,000 ng, or about 600,000 ng, or about 700,000 ng, or about 800,000 ng, or about 900,000 ng. In one example, the amount of gDNA extracted from buffy coat is 10 ng to 300,000 ng, or from 50 ng to 250,000 ng, or from 100 ng to 200,000 ng, or from 150 ng to 150,000 ng, or from 200 ng to 100,000 ng, or from 250 ng to 50,000 ng, or about 10 ng, or about 20 ng, or about 30 ng, or about 40 ng, or about 50 ng, or about 60 ng, or about 70 ng, or about 80 ng, or about 90 ng, or about 100 ng, or about 110 ng, or about 120 ng, or about 130 ng, or about 140 ng, or about 150 ng, or about 160 ng, or about 170 ng, or about 180 ng, or about 190 ng, or about 200 ng, or about 220 ng, or about 230 ng, or about 240 ng, or about 250 ng, or about 260 ng, or about 270 ng, or about 280 ng, or about 290 ng, or about 300 ng, or about 400 ng, or about 500 ng, or about 600 ng, or about 700 ng, or about 800 ng, or about 900 ng, or about 1,000 ng, or about 10,000 ng, or about 100,000 ng, or about 200,000 ng, or about 300,000 ng. In one example, the cfDNA is extracted from the biological sample using a kit such as, but not limited to Zymo Quick-cfRNA Scrum & Plasma Kit (Zymo Research), NextPrep™ Magnazol™ cfRNA Isolation Kit (PerkinElmer), Isopure Plasma cfDNA/RNA Isolation Kit (Aline Biosciences), QIAmp ccfDNA/RNA Kit (Qiagen), QIAmp Circulating Nucleic Acid Kit (Qiagen), EZ1&2 ccfDNA Kit (Qiagen), etc. In one example, the gDNA is extracted from the biological sample using a kit such as, but not limited to MagMAX™ Cell-Free Total Nucleic Acid Isolation Kit (Applied Biosystems), Maxwell® RSC ccfDNA Plasma Kit (Promega), Qiagen DNeasy Blood & Tissue Kit or EZ1&2 DNA Blood Kit (Qiagen), Wizard Genomic DNA Purification Kit (Promega), PureLink Genomic DNA Mini Kit (Invitrogen), etc. It is envisaged that any commercially available kits may be used for such extraction.

In one example, the method of the present disclosure comprises performing a plurality of multiplexed PCR reactions on the extracted nucleic acid as defined in step (b) of the method of the first aspect. In one example, the target nucleic acid is captured by PCR using a highly multiplexed pool of target capture primers. In one example, the method of the present disclosure comprises performing a plurality of multiplexed PCR reactions on the extracted nucleic acid using:
a plurality of target capture primer pairs specific to a plurality of target genes comprising one or more cancer-associated alteration,
wherein each target capture primer pair comprises a forward target capture primer and a reverse target capture primer specific to a target gene comprising one or more cancer-associated alteration,
wherein each forward target capture primer and each reverse target capture primer comprises a target-specific sequence at its 3′ end that is specific to the target gene, a barcode sequence linker, and an adapter-specific sequence at its 5′ end,
thereby generating a plurality of amplicons comprising a plurality of target gene sequences corresponding to the plurality of target genes comprising one or more cancer-associated alteration.

In one example, each forward target capture primer comprises three regions; a partial Illumina sequencing adapter on the 5′ end, a target-specific sequence comprising 15 to 70 nucleotides on the 3′ end to enable hybridisation with target DNA, and a linking barcode sequence comprised of 10 random nucleotides. In one example, each reverse target capture primer comprises three regions; a partial Illumina sequencing adapter on the 5′ end, a target-specific sequence comprising 15 to 70 nucleotides on the 3′ end to enable hybridisation with target DNA, and a linking barcode sequence comprised of 10 random nucleotides.

In one example, the length of the target-specific sequence is from 15 nucleotides to 70 nucleotides, or from 16 nucleotides to 69 nucleotides, or from 17 nucleotides to 68 nucleotides, or from 18 nucleotides to 67 nucleotides, or from 19 nucleotides to 66 nucleotides, or from 20 nucleotides to 65 nucleotides, or from 21 nucleotides to 64 nucleotides, or from 22 nucleotides to 63 nucleotides, or from 23 nucleotides to 62 nucleotides, or from 24 nucleotides to 61 nucleotides, or from 25 nucleotides to 60 nucleotides, or from 26 nucleotides to 59 nucleotides, or from 27 nucleotides to 58 nucleotides, or from 28 nucleotides to 57 nucleotides, or from 29 nucleotides to 56 nucleotides, or from 30 nucleotides to 55 nucleotides, or from 31 nucleotides to 54 nucleotides, or from 32 nucleotides to 53 nucleotides, or from 33 nucleotides to 52 nucleotides, or from 34 nucleotides to 51 nucleotides, or from 35 nucleotides to 50 nucleotides, or from 36 nucleotides to 49 nucleotides, or from 37 nucleotides to 48 nucleotides, or from 38 nucleotides to 47 nucleotides, or from 39 nucleotides to 46 nucleotides, or from 40 nucleotides to 45 nucleotides, or from 41 nucleotides to 44 nucleotides, or 15 nucleotides, or 16 nucleotides, or 17 nucleotides, or 18 nucleotides, or 19 nucleotides, or 20 nucleotides, or 21 nucleotides, or 22 nucleotides, or 23 nucleotides, or 24 nucleotides, or 25 nucleotides, or 26 nucleotides, or 27 nucleotides, or 28 nucleotides, or 29 nucleotides, or 30 nucleotides, or 31 nucleotides, or 32 nucleotides, or 33 nucleotides, or 34 nucleotides, or 35 nucleotides, or 36 nucleotides, or 37 nucleotides, or 38 nucleotides, or 39 nucleotides, or 40 nucleotides, or 41 nucleotides, or 42 nucleotides, or 43 nucleotides, or 44 nucleotides, or 45 nucleotides, or 46 nucleotides, or 47 nucleotides, or 48 nucleotides, or 49 nucleotides, or 50 nucleotides, or 51 nucleotides, or 52 nucleotides, or 53 nucleotides, or 54 nucleotides, or 55 nucleotides, or 56 nucleotides, or 57 nucleotides, or 58 nucleotides, or 59 nucleotides, or 60 nucleotides, or 61 nucleotides, or 62 nucleotides, or 63 nucleotides, or 64 nucleotides, or 65 nucleotides, or 66 nucleotides, or 67 nucleotides, or 68 nucleotides, or 69 nucleotides, or 70 nucleotides. In one example, the length of the target-specific sequence is from 17 nucleotides to 40 nucleotides.

In one example, the barcode sequence is an oligonucleotide comprising 8 to 30 random nucleotides. In one example, the barcode sequence is an oligonucleotide comprising 9 to 29 random nucleotides. In one example, the barcode sequence is an oligonucleotide comprising 10 to 28 random nucleotides. In one example, the barcode sequence is an oligonucleotide comprising 8 random nucleotides. In one example, the barcode sequence is an oligonucleotide comprising 9 random nucleotides. In one example, the barcode sequence is an oligonucleotide comprising 10 random nucleotides. In one example, the barcode sequence is an oligonucleotide comprising 11 random nucleotides. In one example, the barcode sequence is an oligonucleotide comprising 12 random nucleotides. In one example, the barcode sequence is an oligonucleotide comprising 13 random nucleotides. In one example, the barcode sequence is an oligonucleotide comprising 14 random nucleotides. In one example, the barcode sequence is an oligonucleotide comprising 15 random nucleotides. In one example, the barcode sequence is an oligonucleotide comprising 16 random nucleotides. In one example, the barcode sequence is an oligonucleotide comprising 17 random nucleotides. In one example, the barcode sequence is an oligonucleotide comprising 18 random nucleotides. In one example, the barcode sequence is an oligonucleotide comprising 19 random nucleotides. In one example, the barcode sequence is an oligonucleotide comprising 20 random nucleotides. In one example, the barcode sequence is an oligonucleotide comprising 21 random nucleotides. In one example, the barcode sequence is an oligonucleotide comprising 22 random nucleotides. In one example, the barcode sequence is an oligonucleotide comprising 23 random nucleotides. In one example, the barcode sequence is an oligonucleotide comprising 24 random nucleotides. In one example, the barcode sequence is an oligonucleotide comprising 25 random nucleotides. In one example, the barcode sequence is an oligonucleotide comprising 26 random nucleotides. In one example, the barcode sequence is an oligonucleotide comprising 27 random nucleotides. In one example, the barcode sequence is an oligonucleotide comprising 28 random nucleotides. In one example, the barcode sequence is an oligonucleotide comprising 29 random nucleotides. In one example, the barcode sequence is an oligonucleotide comprising 30 random nucleotides. In one specific example, the barcode sequence is an oligonucleotide comprising 10 random nucleotides which can be represented as NNNNNNNNNN (SEQ ID NO: 1).

In one example, the plurality of multiplexed PCR reactions performed on the nucleic acid in step (b) of the method of the first aspect comprises a first PCR step comprising 3 to 15 PCR cycles and the amplification of the plurality of amplicons in the purified mixture as defined in step (e) of the disclosed method comprises a second PCR step comprising 4 to 32 PCR cycles. In one example, the plurality of multiplexed PCR reactions performed on the nucleic acid in step (b) of the method of the first aspect comprises a first PCR step. In one example, the first PCR comprises a low number of PCR cycles for target capture and limited amplification. In one example, the plurality of multiplexed PCR reactions performed on the nucleic acid in step (b) of the method of the first aspect comprises a first PCR step comprising 3 to 15 PCR cycles. In one specific example, the first PCR step comprises 3 to 5 PCR cycles. In one example, the amplification of the plurality of amplicons in the purified mixture as defined in step (e) of the disclosed method comprises a second PCR step. In one example, the amplification of the plurality of amplicons in the purified mixture as defined in step (c) of the disclosed method comprises a second PCR step comprising 4 to 32 PCR cycles. In another specific example, the second PCR step comprises 14 to 16 PCR cycles. In one example, the plurality of amplicons in the purified mixture as defined in step (e) of the disclosed method is subjected to an indexing PCR step (i.e., the second PCR step) where further DNA amplification occurs to increase yields for sequencing, and where predetermined index sequences are added to each amplicon to facilitate subsequent post-sequencing demultiplexing. In one example, the number of PCR cycles in the first PCR step and the second PCR step are independent of each other. In another example, the first PCR step comprises 3 to 5 PCR cycles and the second PCR step comprises 14 to 16 PCR cycles.

In one example, the method of the present disclosure comprises removing target capture primers that have not been incorporated into the plurality of amplicons generated in step (b) of the method of the first aspect using at least one enzyme capable of removing unincorporated target capture primers, for example by degrading the primers via cleavage of phosphodiester bonds between nucleotides of the primers, to thereby generate a purified mixture of the plurality of amplicons comprising the plurality of target gene sequences. In one example, the enzyme is a nuclease. In one example, the method of the present disclosure comprises removing target capture primers that have not been incorporated into the plurality of amplicons generated in step (b) of the method of the first aspect using at least one nuclease, thereby generating a purified mixture of the plurality of amplicons comprising the plurality of target gene sequences. In one example, the nuclease is an endonuclease. In one example, the nuclease is an exonuclease. In one example, the target capture primers to be removed are single-stranded DNA sequences. In one example, the target capture primers are single-stranded DNA primers. In one example, the target capture primers are excess unincorporated single-stranded DNA primers. In one example, the target capture primers are excess single-stranded DNA primers, which remain unincorporated after the target capture step of the multiplexed PCR reactions as defined in step (b) of the first aspect. In one example, the exonuclease may be a single-stranded RNA specific exonuclease.

In one example, the target capture primers that have not been incorporated into amplicons are removed using nucleases selected from the group consisting of exonuclease I, exonuclease T, exonuclease VII, mung bean nuclease (an endonuclease), nuclease P1 (an endonuclease) and nuclease S1 (an endonuclease). In one example, the target capture primers that have not been incorporated into amplicons are removed using: (i) one or more endonuclease, wherein the endonuclease is selected from the group consisting of mung bean nuclease, nuclease P1, nuclease S1 and combinations thereof; or (ii) one or more exonuclease, wherein the exonuclease is selected from the group consisting of exonuclease I, exonuclease T, exonuclease VII and combinations thereof. In one example, the target capture primers that have not been incorporated into amplicons are removed using either exonuclease I, or exonuclease T, or a combination of both exonuclease I and exonuclease T. In one example, the target capture primers that have not been incorporated into amplicons are removed using exonuclease I. In one example, the target capture primers that have not been incorporated into amplicons are removed using exonuclease T. In one example, the target capture primers that have not been incorporated into amplicons are removed using a combination of both exonuclease I and exonuclease T. In one example, following the initial PCR step of the method of the present disclosure, comprising of a low number of PCR cycles (3 to 5 cycles) for target capture and limited amplification, excess target capture primers are removed by treatment with a blend of two exonucleases, exonuclease I and exonuclease T. In one example, the two exonucleases specifically digest unincorporated single-stranded primers in the 3′ to 5′ direction, while retaining double-stranded DNA products containing target regions of interest.

In one example, the method of the present disclosure comprises amplifying the plurality of amplicons in the purified mixture as defined in step (c) of the disclosed method by using universal indexed adapter primers to generate a sequencing library, wherein each amplicon of the sequencing library comprises two barcode sequences. In one example, the sequencing library generated comprises at least at least 1 amplicon, or at least 50 amplicons, or at least 100 amplicons, or at least 200 amplicons, or at least 300 amplicons, or at least 400 amplicons, or at least 500 amplicons, or at least 600 amplicons, or at least 700 amplicons, or at least 800 amplicons, or at least 900 amplicons, or at least 1000 amplicons, or at least 1100 amplicons, or at least 1200 amplicons, or at least 1300 amplicons, or at least 1400 amplicons, or at least 1500 amplicons, or at least 1600 amplicons, or at least 1700 amplicons, or at least 1800 amplicons, or at least 1900 amplicons, or at least 2000 amplicons, or at least 2100 amplicons, or at least 2200 amplicons, or at least 2300 amplicons, or at least 2400 amplicons, or at least 2500 amplicons, or at least 2600 amplicons, or at least 2700 amplicons, or at least 2800 amplicons, or at least 2900 amplicons, or at least 3000 amplicons. In one specific example, the sequencing library generated comprises at least 2600 amplicons. In one example, there is no upper limit for the number of amplicons in the generated sequencing library. In one example, only amplicons comprising barcode sequences and adapters on both their 5′ and 3′ ends are subjected to multiplex sequencing on a next-generation sequencing platform as defined in step (c) of the method of the first aspect. In one example, the sequencing library is subjected to an indexing PCR step where 1) further DNA amplification occurs to increase yields for sequencing and 2) predetermined index sequences are added to each sample to facilitate subsequent post-sequencing demultiplexing. In one example, final libraries with complete structures required for sequencing on the Illumina platform are then pooled together for subsequent sequencing, after which the data is analysed using a bioinformatics pipeline.

In one example, the amplification is performed using KAPA Hifi HotStart ReadyMix (Roche), Phusion U Hot Start DNA Polymerase (Thermo Scientific), ZymoTaq DNA Polymerase (Zymo Research) and Q5U Hot Start High-Fidelity DNA Polymerase (NEB), etc.

In one example, each universal indexed adapter primer as disclosed in step (e) comprises an adapter sequence. In one example, the term “adapter sequence” refers to an oligonucleotide sequence bound to the 5′ and 3′ end of each DNA fragment in a sequencing library. The adapter sequences are complementary to the plurality of oligonucleotides present on the surface of the flow cells of the sequencing tools thereby allowing the DNA fragment to attach to the sequencing tool. In some examples, an adapter sequence allows for the sequencing of the oligonucleotide of interest. Sequencing platform specific adapter sequences are known in the art, and include, for example, the Illumina P5/P7 adapter sequences.

In one example, the universal indexed adapter primers as disclosed in step (c) of the method of the first aspect comprise:

a forward primer comprising the sequence of
(SEQ ID NO: 2)
AATGATACGGCGACCACCGAGATCTACACCTAGCGCTACACTCTTTCCC
TACACGACGCTCTTCCGATC*T;
and
a reverse primer comprising the sequence of
(SEQ ID NO: 3)
CAAGCAGAAGACGGCATACGAGATAACCGCGGGTGACTGGAGTTCAGAC
GTGTGCTCTTCCGATC*T,

wherein “*” represents a phosphorothioate bond, and wherein the underlined sequences are the barcode sequences.

In one example, the method of the present disclosure comprises purifying the sequencing library obtained from step (c) of the disclosed method. In one example, the purification of the plurality of sequencing library is performed using an agent such as paramagnetic beads. In one example, the paramagnetic beads are selected from the group consisting of AMPure XP beads, SPRI beads, and Dynabeads. In one specific example, the sequencing library is purified with two rounds of 0.8× volume AMPure XP beads to remove excess adapters and to size-select the final sequencing library.

In one example, the method of the present disclosure comprises subjecting the purified sequencing library from step (f) of the disclosed method to multiplex sequencing on a next-generation sequencing platform to obtain a plurality of sequencing reads. In one example, each final purified library was qualified using the High Sensitivity DNA Sercentapc (Agilent) and quantified using KAPA Library Quantification Kit (Roche) before being sequenced on an NGS platform. In one example, suitable kits for qualifying and quantifying the final purified library include NEBNext Library Quant Kit (New England Biolabs), Collibri Library Quantification Kit (Thermo Fisher) and ProNex NGS Library Quant Kit (Promega). In some examples, the NGS platform is NextScq 550, NextScq 2000, NovaScq 6000, BGI MGISEQ-2000, DNBSEQ-G400 or DNBSEQ-T7.

In one example, the method of the present disclosure further comprises mapping the plurality of sequencing reads obtained from step (c) of the method of first aspect to a first reference genome. In one example, the term “mapping” refers to the process of aligning sequencing reads to a reference genome. In one example, the term “reference genome” refers to DNA sequences known in the art that may be obtainable from public databases. In one example, the method of the present disclosure further comprises grouping

sequencing reads where the barcode sequences of the sequencing reads are identical into a consensus cluster. The term “consensus cluster” refers to a group of consensus sequence reads wherein at least one of the two barcode sequences of the consensus sequence reads are identical. In one example, the term “consensus cluster” refers to a group of consensus sequence reads wherein one of the two the barcode sequences of the consensus sequence reads are identical. In one example, the term “consensus cluster” refers to a group of consensus sequence reads wherein the two barcode sequences of the consensus sequence reads are identical.

In one example, each consensus cluster as defined in step (h) of the disclosed method comprises at least 1 family member. In one example, each consensus cluster as defined in step (h) of the disclosed method comprises at least 2 family members. In one example, each consensus cluster as defined in step (h) of the disclosed method comprises at least 3 family members. In one example, each consensus cluster as defined in step (h) of the disclosed method comprises at least 4 family members. In one example, each consensus cluster as defined in step (h) of the disclosed method comprises at least 5 family members. In one example, each consensus cluster as defined in step (h) of the disclosed method comprises at least 6 family members. In one example, each consensus cluster as defined in step (h) of the disclosed method comprises at least 7 family members. In one example, each consensus cluster as defined in step (h) of the disclosed method comprises at least 8 family members. In one example, each consensus cluster as defined in step (h) of the disclosed method comprises at least 9 family members. In one example, each consensus cluster as defined in step (h) of the disclosed method comprises at least 10 family members. In one example, there is no upper limit for the number of family members in each consensus cluster as defined in step (h) of the disclosed method. In one example, the method of the present disclosure further comprises performing a second sequence alignment of each consensus cluster obtained from step (h) with all consensus clusters having at least one overlapping barcode sequence. In one example, the term “overlapping barcode sequence” refers to a common barcode sequence between consensus sequencing reads with at least one identical barcode sequence. In one example, prior to performing step (i) of the disclosed method, consensus clusters with fewer than 1 to 5 members are considered unreliable and removed prior to downstream analyses. In one example, prior to performing step (i) of the disclosed method, consensus clusters with fewer than 5 family members are removed from downstream analysis. In one example, prior to performing step (i) of the disclosed method, consensus clusters with fewer than 4 family members are removed from downstream analysis. In one example, prior to performing step (i) of the disclosed method, consensus clusters with fewer than 3 family members are removed from downstream analysis. In one example, prior to performing step (i) of the disclosed method, consensus clusters with fewer than 2 family members are removed from downstream analysis.

In one example, prior to performing step (g) of the disclosed method (I) bases having poor quality scores are removed, and (II) universal indexed adapter primers and barcode sequences are trimmed. In one example, the bases having poor quality scores are removed by replacing the bases with “N”. In one example, the universal indexed adapter primers and barcode sequences are trimmed using bioinformatic tools. In one example, the universal indexed adapter primers and barcode sequences are trimmed using Cutadapt.

In one example, the mapping in steps (g) and (j) and the sequence alignment in step (i) of the disclosed method are performed using bioinformatic algorithms. In one example, the mapping in steps (g) and (j) of the disclosed method is performed using bioinformatic algorithms. In one example, the sequence alignment in step (i) of the disclosed method is performed using bioinformatic algorithms. In one example, the mapping in steps (g) and (j) arc performed using bwa-mem. It is known in the art that Burrows-Wheeler Alignment tool (BWA) is a software package for mapping low-divergent sequences against a large reference genome, such as the human genome. In one example, the sequence alignment in step (i) is performed using MAFFT.

In one example, the method of the present disclosure further comprises mapping each of the consensus sequencing read from step (i) of the disclosed method with a second reference genome.

In one example, the first reference genome as defined in step (g) of the disclosed method and the second reference genome as defined in step (j) of the disclosed method comprise identical gene sequences.

In one example, the method of the present disclosure further comprises identifying the differences between the sequencing read and the reference genome from step (j) of the disclosed method to identify consensus sequencing reads comprising one or more cancer-associated alteration.

In one example, the cancer-associated alterations detected are present at distinct sites in the subject's body.

In one example, each of the one or more pre-determined reference cancer-associated alteration profile as defined in step (d) of the method of the first aspect correlates with one or more cancer type.

In one example, the method of the present disclosure comprises simultaneously identifying presence of one or more cancer type in the subject.

In one example, the one or more cancer type is an early-stage cancer, localized cancer or metastatic cancer. As used herein, early-stage cancer refers to a cancer that is at an initial stage of development and has not spread beyond its site of origin. As used herein, localized cancer refers to a stage of cancer in which the disease is confined to a specific area and has not spread to surrounding tissues or distant organs. As used herein, metastatic cancer occurs when cancer cells have spread from the primary tumor to other parts of the body, forming secondary tumors at distant sites.

In one example, the one or more cancer is a solid tumor or a hematological cancer. As used herein, solid tumor refers to a mass or lump of tissue that consists of abnormal cells, and it can be located in various organs or tissues of the body. As used herein, hematological cancer refers to a cancer which originates in blood-forming tissue, such as the bone marrow, lymphatic system, and/or a blood cell.

In one example, the one or more cancer is selected from the group consisting of lung cancer, liver cancer, breast cancer, colorectal cancer, prostate cancer, nasopharyngeal cancer, pancreas cancer, bile duct cancer, acute myeloid leukemia, chronic myeloid leukemia, head and neck cancer, oropharynx cancer, skin cancer, eye cancer, urinary tract cancer, lymphoplasmacytic lymphoma, cervix cancer, nasopharyngeal cancer, stomach cancer, myeloproliferative neoplasm, endometrium cancer, lymphoma, kidney cancer, and gastrointestinal stromal tumour.

In one example, the subject undergoing the screening/assay disclosed herein does not display symptoms of cancer, i.e. the subject is asymptomatic. In one example, the subject undergoing the screening/assay disclosed herein has not previously been diagnosed with cancer, and/or does not have any known active cancer diagnosis. In one example, the subject undergoing the screening/assay disclosed herein neither display symptoms of cancer nor was previously diagnosed with cancer.

In one example, computation of the one or more likelihood score as defined in step (d) of the method of the first aspect is determined using data from a public database, data from a combination of public databases, curation of data for genetic alterations including the presence of viral DNA, or a combination thereof. In one example, computation of the one or more likelihood score as defined in step (d) of the method of the first aspect is determined using data from one public database. In one example, computation of the one or more likelihood score as defined in step (d) of the method of the first aspect is determined using data from a combination of public databases. In one example, computation of the one or more likelihood score as defined in step (d) of the method of the first aspect is determined using publicly available datasets such as eBioPortal. In one example, computation of the one or more likelihood score as defined in step (d) of the method of the first aspect is determined using expert curation of data for alterations. In one example, expert curation of data for alterations includes alteration with presence of viral DNA. In one example, computation of the one or more likelihood score as defined in step (d) of the method of the first aspect is determined using a combination of data from one public database, data from a combination of public databases, and curation of data for genetic alterations including the presence of viral DNA. In one example, computation of the one or more likelihood score as defined in step (d) of the method of the first aspect is determined using a combination of data from one public database, and data from a combination of public databases. In one example, computation of the one or more likelihood score as defined in step (d) of the method of the first aspect is determined using a combination of data from one public database, and curation of data for genetic alterations including the presence of viral DNA. In one example, computation of the one or more likelihood score as defined in step (d) of the method of the first aspect is determined using a combination of data from a combination of public databases, and curation of data for genetic alterations including the presence of viral DNA.

In one example, the present disclosure refers to a method of detecting one or more cancer-associated alterations and/or determining site of origin of a cancer-associated alteration within a biological sample comprising nucleic acids, comprising the steps of:
A. extracting cell-free nucleic acid and genomic nucleic acid from the biological sample obtained from a subject;
B. performing a plurality of multiplexed PCR reactions on the cell-free nucleic acid and genomic nucleic acid using:
   a plurality of target capture primer pairs specific to a plurality of target genes comprising one or more cancer-associated alteration,
   wherein each target capture primer pair comprises a forward target capture primer and a reverse target capture primer specific to a target gene comprising one or more cancer-associated alteration,
   wherein each forward target capture primer and each reverse target capture primer comprises a target-specific sequence at its 3′ end that is specific to the target gene, a barcode sequence linker, and an adapter-specific sequence at its 5′ end,
   thereby generating a plurality of amplicons comprising a plurality of target gene sequences corresponding to the plurality of target genes comprising one or more cancer-associated alteration;
C. removing target capture primers that have not been incorporated into the plurality of amplicons generated in step B using at least one nuclease, thereby generating a purified mixture of the plurality of amplicons comprising the plurality of target gene sequences;
D. amplifying the plurality of amplicons in the purified mixture obtained from step C by using universal indexed adapter primers to generate a sequencing library, wherein each amplicon of the sequencing library comprises two barcode sequences;
E. purifying the sequencing library obtained from step D;
F. subjecting the purified sequencing library from step E to multiplex sequencing on a next-generation sequencing platform to obtain a plurality of sequencing reads;
G. mapping the plurality of sequencing reads obtained from step F to a first reference genome;
H. grouping the sequencing reads where the barcode sequences of the sequencing reads are identical into a consensus cluster;
I. performing a sequence alignment of each consensus cluster obtained from step H with all consensus clusters having at least one overlapping barcode sequence, then determining the presence of a consensus base in each sequence alignment result to generate a consensus sequencing read;
J. mapping each of the consensus sequencing read from step I with a second reference genome;
K. identifying the differences between the consensus sequencing read and the reference genome from step I to identify consensus sequencing reads comprising one or more cancer-associated alteration;
L. comparing the consensus sequencing reads comprising one or more cancer-associated alteration with one or more pre-determined reference cancer-associated alteration profile to compute one or more likelihood score, wherein the one or more likelihood score indicates the probability of a cancer-associated alteration originating from a specific site of origin,
to thereby detect the presence of one or more cancer-associated alterations and/or determine the site of origin of a cancer-associated alteration.

In one example, step (b) and/or step (c) of the methods of the present disclosure may include a method of simultaneously capturing and identifying distinct targets within a DNA sample, wherein the distinct targets comprise a defined target region and an undefined target region, wherein the undefined target region comprises structural variations or rearrangement or fusion, comprising the steps of:
providing a main mixture comprising a plurality of double stranded DNA fragments A, a plurality of double stranded DNA fragments B, a polymerase, a primer A, and a primer B, wherein:
   the double stranded DNA fragment A is a double stranded DNA fragment comprising a part of the defined target region;
   the double stranded DNA fragment B is a double stranded DNA fragment comprising a part of the undefined target region;
   the primer A comprises, a barcode sequence, and a target-specific sequence A,
      wherein the target-specific sequence A is an oligonucleotide complementary to a sequence at/close to the 3′ end of a single strand of the double stranded DNA fragment A;
   and
   the primer B comprises a separation molecule, a barcode sequence, and a target-specific sequence B,
      wherein the target-specific sequence B is an oligonucleotide complementary to a sequence within a single strand of the double stranded DNA fragment B,
denaturing the double stranded DNA fragment A and the double stranded DNA fragment B thereby allowing the primer A to anneal to a single stranded DNA fragment A and the primer B to anneal to the single stranded DNA fragment B;
allowing the polymerase to elongate the primer A and the primer B thereby obtaining a double stranded product A and a double stranded product B, wherein:
   the double stranded product A is a single stranded elongated primer A that is annealed to the single stranded DNA fragment A; and
   the double stranded product B is a single stranded elongated primer B that is annealed to the single stranded DNA fragment B;
adding a bead that binds the separation molecule in the main mixture and allowing the separation molecule in the double stranded product B to bind to the bead thereby forming a double stranded complex B;
separating the double stranded product A and the double stranded complex B in the main mixture thereby obtaining a mixture A and a mixture B, wherein:
   the mixture A comprises the double stranded product A and
   the mixture B comprises the double stranded complex B;
adding a primer C to the mixture A, wherein the primer C comprises a target-specific sequence C,
   wherein the target-specific sequence C is an oligonucleotide complementary to a sequence at/close to the 3′ end of the single stranded elongated primer A;
denaturing the double stranded product A in the mixture A thereby allowing the primer C to anneal to the single stranded elongated primer A;
allowing the polymerase to elongate the primer C thereby obtaining a double stranded product C, wherein the double stranded product C is a single stranded elongated primer C that is annealed to the single stranded elongated primer A;
connecting a single nucleotide to the 3′ end of the single stranded elongated primer B of the double stranded complex B in the mixture B;
adding a double stranded oligonucleotide to the mixture B wherein the double stranded oligonucleotide comprises a nucleotide overhang complementary to the single nucleotide of step i;
ligating the double stranded oligonucleotide to double stranded complex B at the 3′ end of the single stranded elongated primer B and 5′end of the single stranded DNA fragment B thereby obtaining a double stranded product D;
combining the double stranded product C and the double stranded product D;
amplifying the double stranded product C and the double stranded product D thereby obtaining a plurality of amplicons;
sequencing the plurality of amplicons thereby obtaining a plurality of sequencing result;
using the plurality of sequencing results for:
   identifying single nucleotide sequence variations, or small insertions, or small deletions, or copy number alteration, or deletions of homopolymeric regions, or polymorphism, or microsatellite instability within the defined target regions, or
   identifying the structural variations within the undefined target regions, or
   quantifying the number of distinct targets within the DNA sample.

In a second aspect, the present disclosure refers to a kit for detecting one or more cancer-associated alterations and/or determining site of origin of a cancer-associated alteration within a biological sample according to the method disclosed herein, comprising a plurality of target capture primer pairs specific to a plurality of target genes comprising one or more cancer-associated alteration as defined in step 1 (b) of the method of the first aspect, and instructions for use in the method disclosed herein.

In one example, the kit further comprises: a buffer for performing a plurality of multiplexed PCR reactions, a DNA polymerase, a plurality of deoxynucleoside triphosphates (dNTPs), and one or more nucleases capable of removing unincorporated target capture primers. In some examples, the reagents provided in the kit as described herein may be provided in separate containers comprising the components independently distributed in one or more containers. As the method as described herein relates to sequencing (such as high-throughput sequencing), further components required in sequencing process could be easily determined by the person skilled in the art.

As used in this application, the singular form “a,” “an,” and “the” include plural references unless the context clearly dictates otherwise. For example, the term “a primer” includes a plurality of primers, including mixtures and combinations thereof.

As used herein, the terms “increase” and “decrease” refer to the relative alteration of a chosen trait or characteristic in a subset of a population in comparison to the same trait or characteristic as present in the whole population. An increase thus indicates a change on a positive scale, whereas a decrease indicates a change on a negative scale. The term “change”, as used herein, also refers to the difference between a chosen trait or characteristic of an isolated population subset in comparison to the same trait or characteristic in the population as a whole. However, this term is without valuation of the difference seen.

As used herein, the term “about” in the context of concentration of a substance, size of a substance, length of time, or other stated values means+/−5% of the stated value, or +/−4% of the stated value, or +/−3% of the stated value, or +/−2% of the stated value, or +/−1% of the stated value, or +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The present disclosure illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments and optional features, modification and variation of the present disclosure embodied therein herein disclosed may be resorted to by those skilled in the art, and that such modifications and variations are considered to be within the scope of this present disclosure.

The disclosure has been described broadly and generically herein. Each of the narrower species and subgeneric groupings falling within the generic disclosure also form part of the present disclosure. This includes the generic description of the present disclosure with a proviso or negative limitation removing any subject matter from the genus, regardless of whether or not the excised material is specifically recited herein.

Other embodiments are within the following claims and non-limiting examples.

### EXAMPLES

### Materials

Exemplary Molecular Tag Complex or Primers when Target is EGFR_Exon19

An example of a “primer” when the target sequence is EGFR_exon19 is as follows:

(SEQ ID NO: 4)
ACACGACGCTCTTCCGATCTNNNNNNNNNNTGCCAGTTAACGTCTTCCT
TCTC.

wherein the bases in italic and underline are an example of adapter sequence, the bases in bold represent the barcode sequence and the bases in underline is an example of target specific sequence.

An example of subsequent primers for the “completion of amplicon” is as follows:

(SEQ ID NO: 5)
GACGTGTGCTCTTCCGATCTNNNNNNNNNNCCACACAGCAAAGCAGAAA
CTC,

wherein the bases in italic and underline are an example of adapter sequence, the bases in bold represent the barcode sequence and the bases in underline is an example of target specific sequence.

Expected amplicon (only target-specific region)

>chr7: 55242380 + 55242537 158 bp
(SEQ ID NO: 6)
TGCCAGTTAACGTCTTCCTTCTCtctctgtcatagggactctggatcccagaaggtgagaaagttaaaattcccgtc
gctatcaaggaattaagagaagcaacatctccgaaagccaacaaggaaatcctcgatgtGAGTTTCTGCTTTGCTGT
GTGG

Product after amplicon completion (in two steps) (Only one strand of the double stranded product is shown.):

(SEQ ID NO: 7)
ACACGACGCTCTTCCGATCTNNNNNNNNNNTGCCAGTTAACGTCTTCCTTCTCtctct
gtcatagggactctggatcccagaaggtgagaaagttaaaattcccgtcgctatcaaggaattaagagaagcaacatctccgaaagcc
aacaaggaaatcctcgatgtGAGTTTCTGCTTTGCTGTGTGGNNNNNNNNNNAGATCGGAAGAGCACACGTC,

where the bases in underline is target nucleic acid.

### Final Product (Suitable for Sequencing on Illumina)

(SEQ ID NO: 8)
AATGATACGGCGACCACCGAGATCTACACCTAGCGCTACACTCTTTCCCTACACG
ACGCTCTTCCGATCTNNNNNNNNNNTGCCAGTTAACGTCTTCCTTCTCtctctgtcatagg
gactctggatcccagaaggtgagaaagttaaaattcccgtcgctatcaaggaattaagagaagcaacatctccgaaagccaacaagg
aaatcctcgatgtGAGTTTCTGCTTTGCTGTGTGGNNNNNNNNNNAGATCGGAAGAGCAC
ACGTCTGAACTCCAGTCACCCGCGGTTATCTCGTATGCCGTCTTCTGCTTG,

where the bases in underline is target nucleic acid.

### Methods

### Sample Collection and Processing

Blood collected in Cell-free DNA BCT (Streck) was shipped at ambient temperature before plasma separation. Plasma was prepared using a two-step centrifugation process: the first centrifugation was done at 1600×g for 10 min at 4° C. to separate plasma. The plasma layer was then transferred to a separate tube and centrifuged at 16,000×g for 10 min at 4° C. to further remove cellular contaminants, and immediately processed for nucleic acid extraction or stored at −80° C. until used for extraction. If frozen, the plasma was fully thawed at room temperature before extraction.

Cell-free total nucleic acids were extracted from 3 to 5 mL of plasma using the QIAamp Circulating Nucleic Acid kit (Qiagen). cIDNA was quantified using the Qubit 1×dsDNA High Sensitivity kit (Thermo Fisher Scientific).

### Preparation of Sequencing Library

The generation of a sequencing library is achieved in three steps:
1. Barcode sequence assignment and amplicon generation (Multiplex target capture PCR)
2. Removal of excess target capture primers (Exonuclease treatment)
3. Final library amplification (Indexing PCR)

### Barcode Sequence Assignment and Amplicon Generation

In this first step, target DNA molecules are captured with a pair of primers per target. Each target capture primer is composed of three parts—the target-specific sequence, a 10-base pair random nucleotide sequence (NNNNNNNNNN) (SEQ ID NO: 1) upstream of the target-specific sequence, and an adapter-specific sequence. The target-specific sequence achieves target capture, the 10-base pair random nucleotide constitutes the “unique barcode sequence”, and the adapter-specific sequence serves as the primer landing site for the final library amplification primers. The combination of the target-specific sequence and the 10-base pair unique barcode sequence for both forward and reverse primers were used to trace and define a unique original parental DNA molecule.

cfDNA was used as a template in a highly multiplexed PCR reaction for target capture using the Platinum™ SuperFi II DNA Polymerase (Thermo Fisher Scientific). Briefly, in a 50 μl PCR reaction, cfDNA was mixed with target capture primers at a final concentration of 10 to 100 nM (each primer), 10 μl of 5× SuperFi II Buffer, 10 nM dNTPs, and 2 μl Platinum SuperFi II DNA Polymerase, and subjected to the following thermocycling conditions: initial denaturation at 98° C. for 1 minute; followed by 3 to 5 cycles of denaturation at 98° C. for 10 seconds, annealing at 58° C. for 6 minutes, extension at 72° C. for 5 minutes; and lastly a final extension at 72° C. for 5 minutes.

### Removal of Excess Target Capture Primers

The PCR product underwent exonuclease treatment (using exonucleases, ExoI and ExoT) with 10×NEBuffer r3.1 (NEB), followed by an incubation at 37° C. for 10 min. The exonuclease-treated product was then subjected to clean-up using 1.5× volume of AMPure XP beads (Beckman Coulter), and eluted in Buffer EB (Qiagen).

### Final Library Amplification

Purified products were then amplified with universal indexed adapter primers using KAPA HiFi HotStart ReadyMix (Roche). The PCR was carried out with the following thermocycling profile: initial denaturation at 98° C. for 45 seconds; followed by 14 to 16 cycles of denaturation at 98° C. for 15 seconds, annealing at 60° C. for 30 seconds, extension at 72° C. for 30 seconds; and lastly a final extension at 72° C. for 1 minute. The amplified library was purified with two rounds of 0.8× volume AMPure XP beads to remove excess adapters and to size-select the final sequencing library. Each final purified library was qualified using the High Sensitivity DNA Screentape (Agilent) and quantified using KAPA Library Quantification Kit (Roche) before being sequenced on a NextSeq 550 system (Illumina).

### Data Analysis

Binary base call sequencing files were first demultiplexed and converted to FASTQ files, which are processed using a custom pipeline. First, bases with poor quality scores were filtered. Next, read 1 and corresponding read 2 FASTQ files were searched for expected forward and reverse primer sequences respectively, based on an input file containing named primer sequences of all amplicons within the panel. Primer sequences and upstream barcode sequences were trimmed using Cutadapt and the trimmed sequences were mapped to the reference genome using bwa-mem. Reads were annotated with their corresponding primer names. The primer name assigned to read 1 may not always match that of read 2 due to overlapping amplicons or non-specific binding. An “amplicon_name” was assigned to each read pair by concatenating the matching primer name of reads 1 and 2 (F_name;R_name). Barcode sequences from both reads 1 and 2 were also concatenated and assigned separately to each paired read (F_barcode;R_barcode).

Subgraph consensus clustering of barcode sequences was performed by considering each amplicon_name as a network. Each read assigned the same amplicon_name was represented within the amplicon_name network as a subgraph of two connected nodes of identity F_barcode and R_barcode. Every subsequent read was added to the network either as a disconnected subgraph or joined to an existing subgraph via a common barcode (either F_barcode or R_barcode), until no more reads were left. Each consensus cluster was a disconnected subgraph within the network and was represented by the amplicon_name appended with a number (amplicon_name_n), representing the number of disconnected subgraphs for each amplicon. Consensus clusters with fewer than 1 to 5 members were considered unreliable and removed prior to downstream analyses.

Consensus calling was done for each consensus cluster, first via global alignment of all consensus family members using MAFFT. The consensus base in each aligned position was called by determining the majority representative base, the percentage of which was no less than an automatically determined threshold, which is a function of the total number of reads within the consensus cluster. If no representative base can be called, the position was assigned N, as opposed to one of A, C, T, G. A new quality score was assigned to each position, which was either 90th percentile of all the quality values from the representative base type in that position if a consensus base was found, or 10th percentile of all quality values in that position if no consensus base was found. The consensus reads are written to new consensus FASTQ files, which were then mapped to the reference genome with local realignment to improve mapping. Consensus read depth was calculated from the mapped BAM file as the unique number of consensus clusters mapped to each target region specified in the panel. Variant calling was performed on consensus BAM files using a custom variant caller.

### TOO Prediction

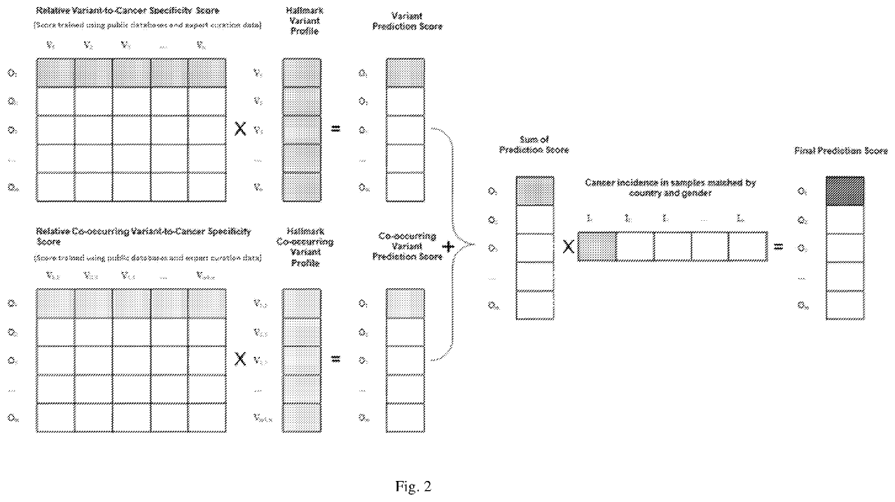

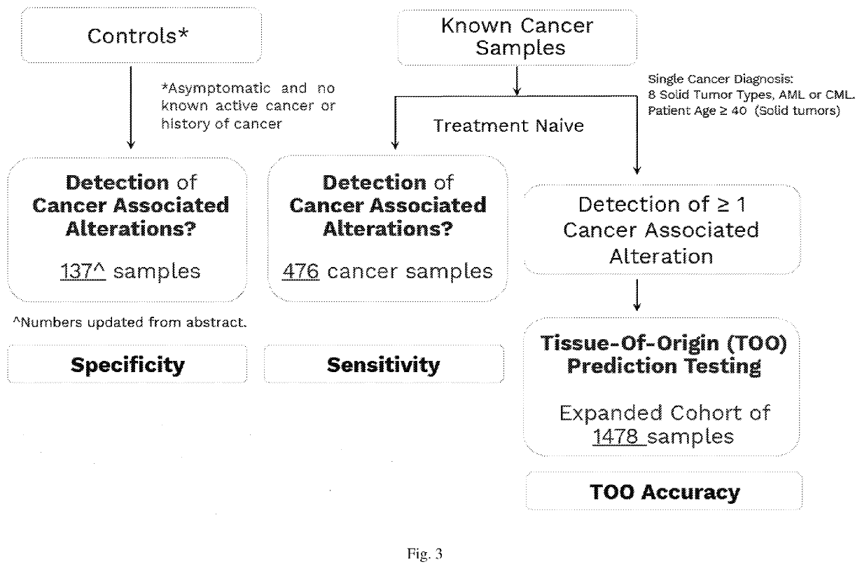

A highly multiplex amplicon-based NGS assay was designed to capture cancer-associated variants, including SNV and indel mutations, as well as viral DNA (EBV, HBV and HPV—20 genotypes). The source of cancer-associated variant could be cfDNA or gDNA, as the method is applicable to both solid tumors and hematological cancers. Primer design was guided by the prevalence and specificity of the mutation in specific cancer types, using the model illustrated in FIG. 2.

The prediction of the tissue of origin of the cancer is performed for the test. An in-house developed model termed the Relative Variant-to-Cancer Specificity Score (RVCSS) was used to predict the primary site of a tumor.

The model was trained using a combination of data from public databases and expert curation of data for alterations including the presence of viral DNA. The RVCSS was calculated in three steps:

### 1. Variant Prevalence in a Specific Cancer Type

The first step calculates the proportion of samples in a specific cancer type that have the variant of interest. This is a straightforward measure of how common the variant is within that cancer type.

$$Variant {Prevalence}_{specific cancer} = Number of samples with the variant in the spcific cancer / Total number of samples in the specific cancer$$

### 2. Relative Variant-to-Cancer Specificity Score (RVCSS)

In the second step, the frequency of the variant was normalized in a specific cancer type by the sum of its frequencies across all cancer types. This step ensures that the score reflects how specific the variant is to a particular cancer type relative to its overall occurrence across all cancers.

$${RVCSS}_{specific cancer} = Variant {Prevalence}_{specific cancer} / {\sum }_{all cancers} Variant {Prevalence}_{specific cancer}$$

### 3. Smoothing

Some cancer types might have no prevalence due to the limited size of the data. The following smooth function was added to help in reducing the bias introduced by small sample size

$${\sum }_{all cancers} Variant {Prevalence}_{specific cancer} + smooth\_prevalence * np . log ( max ( 1 , total\_cancer \_types \_whit \_no \_prevalence ) )$$

$$Smooth\_prevalence = ( {\sum }_{all cancers} 1 / total number of samples in specific cancer ) / total cancer types$$

Finally, the RVCSS of all cancer-associated variants and co-occurring cancer-associated variants in a sample for each cancer type were summed to generate a prediction score. The prediction score for each cancer type was adjusted by country and gender and the final prediction was made based on the two cancer types with the highest scores passing a specific threshold of 0.4.

### Real-World Experience of MCS Assay

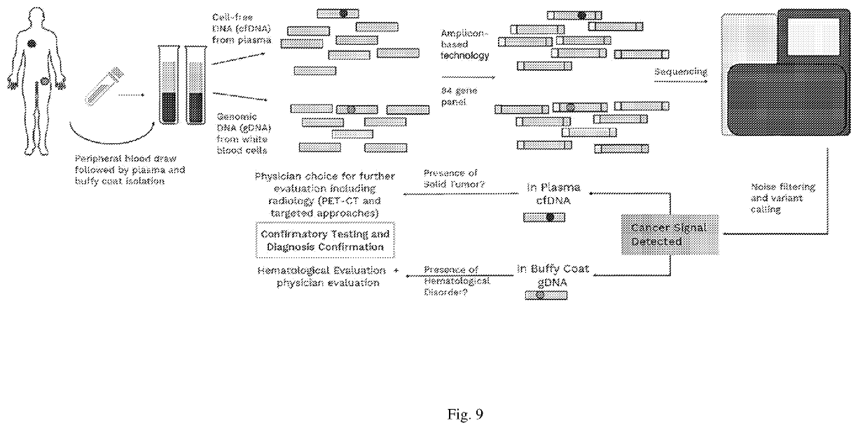

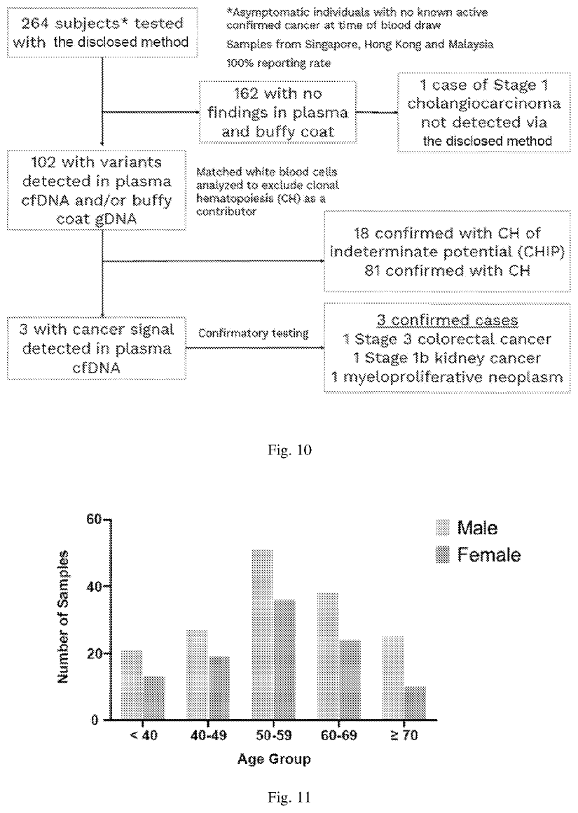

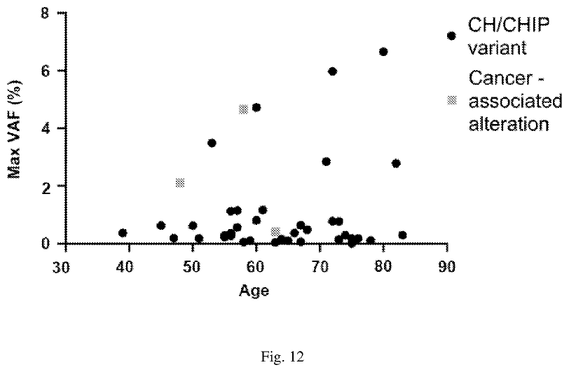

Targeted ultrasensitive amplicon-based NGS with mirror barcodes was used to detect cancel signal (CS) status by detecting cancer-associated mutations and viral DNA in 84 genes in plasma cfDNA. Matched white blood cell DNA was analyzed to exclude clonal hematopoiesis as a contributor (FIG. 9). Consecutive results from 264 subjects (1/2023-6/2023) from Singapore, Hong Kong and Malaysia that underwent testing at a CAP-accredited, CLIA-certified laboratory were included in the analysis (FIG. 10).

### Results

### Sensitivity and Specificity of Cancer-Associated Alterations Detection in MCS Assay

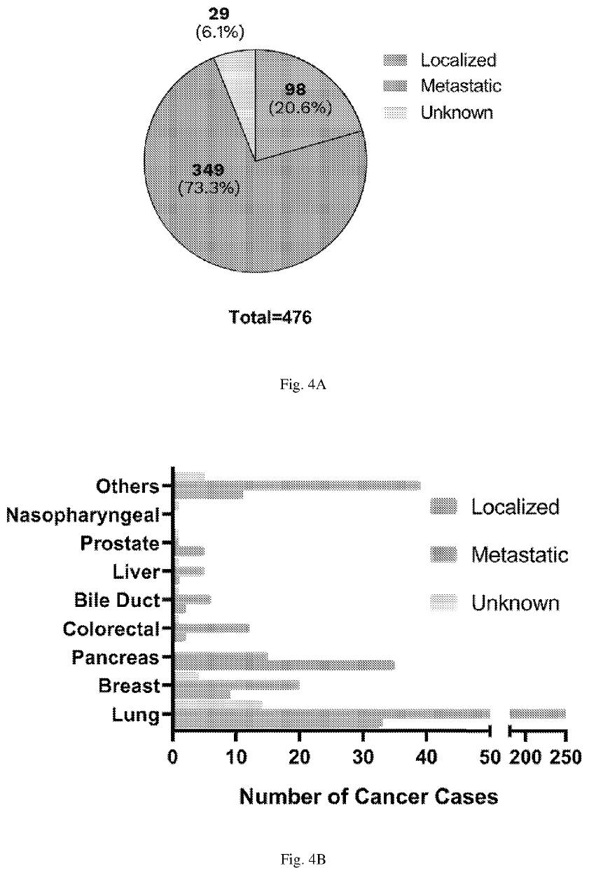

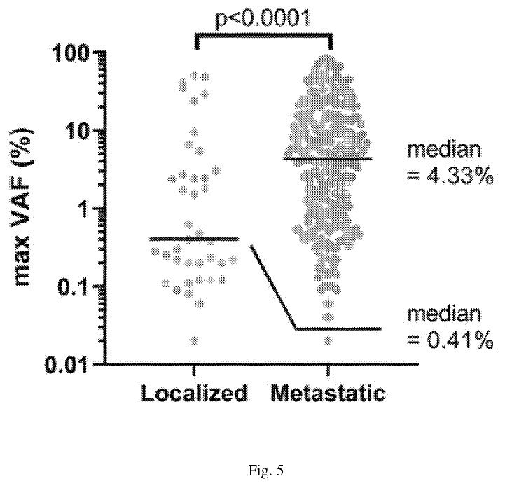

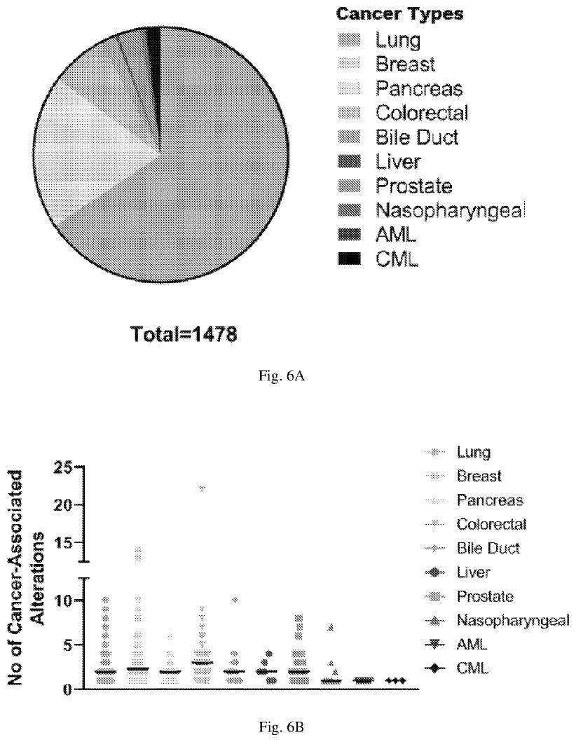

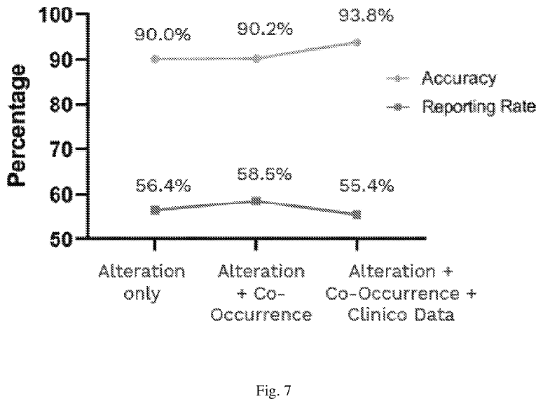

The sensitivity of detection of cancer associated alterations was evaluated in a cohort of 476 untreated cancer patients and the specificity of assay was evaluated in a cohort of 137 controls. A weighted sum model was used for single site TOO prediction in an expanded cohort of 1478 samples with a clear single cancer diagnosis in 10 cancer types (FIG. 4). Weights assigned to each cancer-associated alteration and its co-occurring combinations represent the importance or contribution of each alteration to each potential TOO. The impact of inclusion of clinico-demographic data (where available) on TOO prediction was also evaluated. Overall sensitivity for detection of cancer-associated alterations was 72.7% (346/476). Sensitivity is higher in metastatic cases (82.5%) compared to localized cases (41.8%) (Table 1).

TABLE 1
Table showing higher sensitivity observed in metastatic cases

| | All | Localized | Metastatic | Unknown |
|---|---|---|---|---|
| % of Cases with | 72.7 | 41.8 | 82.5 | 58.6 |
| cancer-associated | (N = | (N = | (N = | (N = |
| alteration detected | 346/476) | 41/98) | 288/349) | 17/29) |

Sample-level specificity for cancer-associated alterations/EBV detection was 96% (48/50), also comparable to previous scries. Cancer-associated alterations/EBV were detected in approximately 50% of localized cases for 3 cancer types with no established screening recommendations in Asia (lung-54.6%; bile duct-50.0%; pancreas-48.6%) (Table 2).

TABLE 2
Table showing detection of cancer-associated alterations
in ~50% of localized cases for lung, bile duct and
pancreas (no established screening recommendations in Asia)

Percentage of Cases with Cancer
Associated Alterations (%)

| Cancer Type | All | Localized | Metastatic |
|---|---|---|---|
| Lung | 79.9 | 54.6 | 83.7 |
| Breast | 57.6 | 22.2 | 75.0 |
| Pancreas | 62.0 | 48.6 | 93.3 |
| Colorectal | 80.0 | Low Sample Count | 91.7 |
| Bile Duct | 88.9 | 50.0 | 100.0 |
| Liver | 28.6 | Low Sample Count | 40.0 |
| Prostate | 42.9 | 40.0 | 100.0 |

### Tissue-Of-Origin (TOO) Prediction

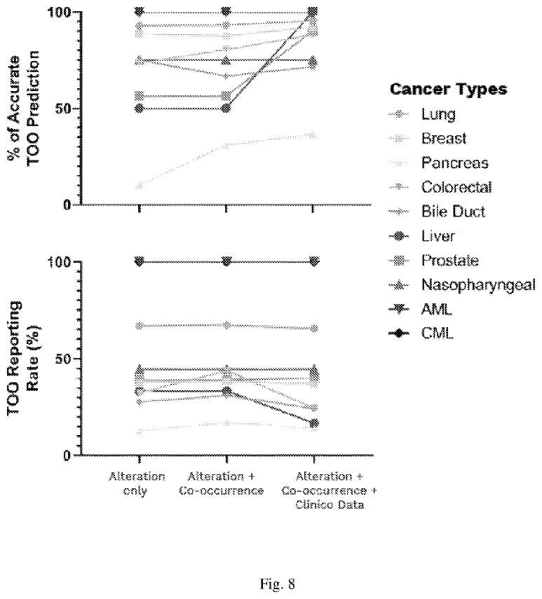

In the expanded cohort, a high confidence call for TOO was obtained in 55.4% (818/1478) of samples with positive findings. Clinico-demographic data inclusion as a model variable improved overall TOO prediction accuracy in 10 cancer types to 93.8% (767/818), compared to an accuracy of 90.2% without factoring in clinico-demographic data. Inclusion of clinico-demographic data as a model variable improved overall TOO prediction accuracy. Improved TOO prediction was observed in seven out of 10 cancer types (improvement of 2.18-50.0%). TOO reporting rate remained largely consistent in most cancer types (FIG. 8).

### Real-World Experience of MCS Assay

Results were returned for 100% of subjects with a mean turnaround time of 14.1 calendar days. Mean age was 55.1 years (yrs) with 61.4% being male (FIG. 11). CS-positive rate was 1.1% (3/264) (Table 3). All 3 CS-positive subjects were male, >40 years of age and were asymptomatic and without known cancer at screening. All 3 subsequently had neoplasms confirmed (locally advanced colon cancer, localized kidney cancer, and myeloproliferative neoplasm), for a specificity and PPV of 100%, 2 of 3 of these cases (kidney and myeloproliferative neoplasm) would not have been covered under traditional screening guidelines. One subject with CS-negative test results had a suspected cholangiocarcinoma at the point of test order, for an overall sensitivity of 75%.

TABLE 3
Table showing number of subjects that were tested positive
for cancer using the MCS assay disclosed herein

| | Cancer Confirmed | No Cancer |
|---|---|---|
| Method Positive | 3 | 0 |
| Method Negative | 1 | 260 |

### Discussion

The present disclosure describes experimental and data analysis workflows which enable simultaneous determination of multiple cancer-associated alterations and determination of the site of origin of a cancer-associated alteration within a biological sample comprising nucleic acids.

The circulating tumor DNA (ctDNA) mutation-based testing workflow described herein showed reasonable sensitivity and specificity for detection of cancer-associated alterations, based on a retrospective study cohort. Tumor TOO can also be correctly classified in the majority of patients. Incorporation of clinico-demographic data to personalize TOO recommendations also improved accuracy of calls for subsequent confirmatory testing and diagnosis confirmation.

The present disclosure provides real-world initial experience data with a cohort undergoing amplicon-based MCED testing in Asia. A majority of cases detected were not previously covered under traditional screening guidelines. These findings provide additional support for the use of ultrasensitive mutation-based assays for screening.

The present disclosure describes for the first time:
1. A multi-cancer screening liquid biopsy testing assay with a custom pipeline incorporating clinico-demographic factors to improve TOO prediction results.
2. The ability to detect with high specificity and sensitivity, multiple cancer-associated alterations and determine the site of origin of a cancer-associated alteration using a single biological sample.
3. The present disclosure uses the cancer-associated alterations described herein (SNV and indel mutations and viral DNA) unlike conventional screening methods for multi-cancer early detection, which typically use a combination of protein and methylation detection methods-both for cancer detection and prediction of tissue-of-origin.

Notable features of the method disclosed herein include:
1. The use of a combination of cfDNA from blood plasma, and gDNA from buffy coat of blood to exclude clonal hematopoiesis for accurate and reliable detection results in cancer diagnostics.
2. The simultaneous determination of the TOO of a tumor using the TOO prediction method disclosed herein to correctly classify the cancer patients for further clinical evaluation including radiology (PET-CT and targeted approaches) or Hematological Evaluation and physician evaluation.
3. The incorporation of clinico-demographic data in the TOO prediction method disclosed herein to personalize TOO recommendations and to improve accuracy of TOO prediction calls.

The method of the present disclosure has the following advantages:
1. The method of the present disclosure allows for early detection of multiple cancers and simultaneous TOO prediction for better cancer prognosis.
2. The method of the present disclosure allows for incorporation of clinico-demographic data to improve accuracy of TOO predictions which is critical for tailoring treatments to the specific characteristics of the cancer.

SEQUENCE LISTING

| SEQ ID | | |
|---|---|---|
| NO. | Sequence Name | Sequence |
| 1 | Barcode sequence | NNNNNNNNNN |
| 2 | Universal indexed adapter | AATGATACGGCGACCACCGAGATCTAC |
| | forward primer | ACCTAGCGCTACACTCTTTCCCTACACG |
| | | ACGCTCTTCCGATC*T |
| 3 | Universal indexed adapter | CAAGCAGAAGACGGCATACGAGATAAC |
| | reverse primer | CGCGGGTGACTGGAGTTCAGACGTGTG |
| | | CTCTTCCGATC*T |
| 4 | Exemplary forward target | ACACGACGCTCTTCCGATCTNNNNNNNN |
| | capture primer if the target | NNTGCCAGTTAACGTCTTCCTTCTC |
| | sequence is EGFR_exon19 | |
| 5 | Exemplary reverse target | GACGTGTGCTCTTCCGATCTNNNNNNNN |
| | capture primer if the target | NNCCACACAGCAAAGCAGAAACTC |
| | sequence is EGFR_exon 19 | |
| 6 | Exemplary expected | TGCCAGTTAACGTCTTCCTTCTCtctctgtcata |
| | amplicon (only target- | Gggactctggatcccagaaggtgagaaagttaaaattcccgtcgc |
| | specific region) | Tatcaaggaattaagagaagcaacatctccgaaagccaacaagga |
| | | aatcctcgatgtGAGTTTCTGCTTTGCTGTGTGG |
| 7 | Exemplary product after | ACACGACGCTCTTCCGATCTNNNNNNNNN |
| | amplicon completion (in | NTGCCAGTTAACGTCTTCCTTCTCtctctgtcata |
| | two steps) | gggactctggatcccagaaggtgagaaagttaaaattcccgtcgcta |
| | | tcaaggaattaagagaagcaacatctccgaaagccaacaaggaaat |
| | | cctcgatgtGAGTTTCTGCTTTGCTGTGTGGNN |
| | | NNNNNNNNAGATCGGAAGAGCACACGTC |
| 8 | Exemplary final product | AATGATACGGCGACCACCGAGATCTACAC |
| | | CTAGCGCTACACTCTTTCCCTACACGACGC |
| | | TCTTCCGATCTNNNNNNNNNNTGCCAGTTA |
| | | ACGTCTTCCTTCTCtctctgtcatagggactctggatccca |
| | | gaaggtgagaaagttaaaattcccgtcgctatcaaggaattaagagaa |
| | | gcaacatctccgaaagccaacaaggaaatcctcgatgtGAGTTT |
| | | CTGCTTTGCTGTGTGGNNNNNNNNNNAGA |
| | | TCGGAAGAGCACACGTCTGAACTCCAGTC |
| | | ACCCGCGGTTATCTCGTATGCCGTCTTCTG |
| | | CTTG |

1. A method of detecting one or more cancer-associated alterations and/or determining site of origin of a cancer-associated alteration within a biological sample comprising nucleic acids, comprising the steps of:
(a) extracting nucleic acid from the biological sample obtained from a subject;
(b) performing a plurality of multiplexed PCR reactions on the nucleic acid using:
a plurality of target capture primer pairs specific to a plurality of target genes comprising one or more cancer-associated alteration,
wherein each target capture primer pair comprises a forward target capture primer and a reverse target capture primer specific to a target gene comprising one or more cancer-associated alteration,
wherein each forward target capture primer and each reverse target capture primer comprises a target-specific sequence at its 3′ end that is specific to the target gene, a barcode sequence linker, and an adapter-specific sequence at its 5′ end,
thereby generating a plurality of amplicons comprising a plurality of target gene sequences corresponding to the plurality of target genes comprising one or more cancer-associated alteration;
(c) sequencing the plurality of amplicons from step (b) to obtain a plurality of sequencing reads, wherein optionally, the sequencing is multiplex sequencing on a next-generation sequencing platform; and
(d) comparing the plurality of sequencing reads comprising one or more cancer-associated alteration with one or more pre-determined reference cancer-associated alteration profile to compute one or more likelihood score, wherein the one or more likelihood score indicates the probability of a cancer-associated alteration originating from a specific site of origin,
to thereby detect the presence of one or more cancer-associated alterations and/or determine the site of origin of a cancer-associated alteration.

2. The method of claim 1, wherein prior to performing step (c), the method comprises further performing a step of removing target capture primers that have not been incorporated into the plurality of amplicons generated in step (b) using at least one nuclease, thereby generating a purified mixture of the plurality of amplicons comprising the plurality of target gene sequences, optionally wherein the at least one nuclease used to remove the target capture primers that have not been incorporated into amplicons is an endonuclease and/or an exonuclease.

3. The method of claim 2, further comprising the steps of:
(e) amplifying the plurality of amplicons in the purified mixture as defined in claim 2 by using universal indexed adapter primers to generate a sequencing library, wherein each amplicon of the sequencing library comprises two barcode sequences;
(f) purifying the sequencing library obtained from step (e);
(g) mapping the plurality of sequencing reads obtained from step (c) of claim 1 to a first reference genome;
(h) grouping the sequencing reads where the barcode sequences of the sequencing reads are identical into a consensus cluster;
(i) performing a sequence alignment of each consensus cluster obtained from step (h) with all consensus clusters having at least one overlapping barcode sequence, then determining the presence of a consensus base in each sequence alignment result to generate a consensus sequencing read;
(j) mapping each of the consensus sequencing read from step (i) with a second reference genome; and
(k) identifying the differences between the consensus sequencing read and the reference genome from step (j) to identify consensus sequencing reads comprising one or more cancer-associated alteration.

4. The method of claim 1, wherein step (d) further comprises optimizing the one or more likelihood score based on a clinico-demographic profile of the subject.

5. The method of claim 1, wherein the nucleic acid is cell-free nucleic acid or genomic nucleic acid, wherein optionally the cell-free nucleic acid is cell-free DNA (cfDNA) or cell-free RNA (cfRNA), wherein optionally the genomic nucleic acid is genomic DNA (gDNA) or genomic RNA (gRNA), and wherein optionally the nucleic acid comprises cfDNA, cfRNA, gDNA, gRNA, or a combination thereof.

6. The method of claim 5, wherein the cfDNA is circulating tumor DNA (ctDNA).

7. The method of claim 1, wherein the one or more cancer-associated alteration is selected from a group comprising of single-nucleotide variations, insertions, deletions, genomic copy number alterations, deletions of homopolymeric regions, total mutation (or variant) load, detection of microbial DNA sequences, detection of polymorphisms or single-nucleotide variations in nucleic acid sequences, DNA structural rearrangements giving rise to variant RNA molecules, and incorporation of viral DNA, wherein optionally the variant RNA molecules are selected from the group consisting of structural variants of RNA molecules and copy number alterations of RNA molecules, wherein optionally the viral DNA is Epstein-Barr virus (EBV), hepatitis B virus (HBV), and/or Human papillomavirus (HPV).

8. The method of claim 1, wherein the biological sample is a liquid sample, optionally wherein the liquid sample is bodily fluid, optionally wherein the bodily fluid is blood, urine, or saliva, and wherein optionally the blood is plasma or buffy coat.

9. The method of claim 1, wherein if the biological sample is blood, then step (a) further comprises separating the biological sample into a plasma component and a buffy coat component, prior to extracting cfDNA or cfRNA from the plasma component and gDNA or gRNA from buffy coat component.

10. The method of claim 9, wherein the plurality of multiplexed PCR reactions as defined in step (b) performed on the plasma component and the buffy coat component are independent of each other.

11. The method of claim 9, wherein the cfDNA and gDNA to be used for step (b) harbor cancer-associated alteration(s) that arise independently from clonal hematopoiesis.

12. The method of claim 1, wherein the cancer-associated alterations detected are present at distinct sites in the subject's body.

13. The method of claim 4, wherein the clinico-demographic profile comprises nationality, gender, age, or a combination thereof.

14. The method of claim 1, wherein each of the one or more pre-determined reference cancer-associated alteration profile as defined in step (d) correlates with one or more cancer type.

15. The method of claim 1, comprising simultaneously identifying presence of one or more cancer type in the subject.

16. The method of claim 14, wherein the one or more cancer type is an early-stage cancer, localized cancer or metastatic cancer.

17. The method of claim 14, wherein the one or more cancer is a solid tumor or a hematological cancer.

18. The method of claim 14, wherein the one or more cancer is selected from the group consisting of lung cancer, liver cancer, breast cancer, colorectal cancer, prostate cancer, nasopharyngeal cancer, pancreas cancer, bile duct cancer, acute myeloid leukemia, chronic myeloid leukemia, head and neck cancer, oropharynx cancer, skin cancer, eye cancer, urinary tract cancer, lymphoplasmacytic lymphoma, cervix cancer, nasopharyngeal cancer, stomach cancer, myeloproliferative neoplasm, endometrium cancer, lymphoma, kidney cancer, and gastrointestinal stromal tumour.

19. The method of claim 1, wherein the subject does not display symptoms of cancer and/or the subject has not previously been diagnosed with cancer.

20. The method of claim 1, wherein computation of the one or more likelihood score as defined in step (d) is determined using data from a public database, data from a combination of public databases, curation of data for genetic alterations including the presence of viral DNA, or a combination thereof.

21. A kit for detecting one or more cancer-associated alterations and/or determining site of origin of a cancer-associated alteration within a biological sample according to the method of claim 1, comprising a plurality of target capture primer pairs specific to a plurality of target genes comprising one or more cancer-associated alteration, and instructions for use in the method of claim 1.

22. The kit according to claim 21, wherein the kit further comprises:
a buffer for performing a plurality of multiplexed PCR reactions,
a DNA polymerase,
a plurality of deoxynucleoside triphosphates (dNTPs), and
a reagent capable of removing excess primers.
